(19) United States
(12) Patent Application Publication
(10) Pub. No.: US 2022/0031979 A1
(43) Pub. Date: Feb. 3, 2022

(54) DEVICE FOR NASAL DELIVERY OF FLUID PRODUCT

(71) Applicant: APTAR FRANCE SAS, LE NEUBOURG (FR)

(72) Inventors: Charles CAZZOLA, ROUEN (FR); Nicolas NARBONE, GRAVIGNY (FR)

(73) Assignee: APTAR FRANCE SAS, LE NEUBOURG (FR)

(21) Appl. No.: 17/276,958

(22) PCT Filed: Sep. 17, 2019
(86) PCT No.: PCT/FR2019/052157

§ 371 Date: Mar. 17, 2021

(30) Foreign Application Priority Data
Sep. 19, 2018 (FR) FR1858448

(51) Int. Cl. A61M 15/08; A61M 11/00

(52) U.S. Cl. CPC A61M 15/08; A61M 2202/064; A61M 2205/073; A61M 11/007

(57) ABSTRACT

A nasal fluid dispenser device comprising: a body (10) comprising a hollow sleeve (11) and a top surface (15); and an actuator member (40) that is mounted to slide in said body (10) so as to actuate the device; the device further comprising two nasal extensions (20, 30), at least one of which forms a dispenser head provided with a respective dispenser orifice (21, 31), said nasal extensions extending from said top surface (15), each dispenser head being connected, during actuation, to a respective reservoir (50) that is arranged in said body (10), said two nasal extensions (20, 30) forming an angle lying in the range 30° to 60° relative to a longitudinal central axis (A) of said body (10).

The present invention relates to a nasal fluid dispenser device.

Nasal dispenser devices are well known. They generally comprise a reservoir containing one or more doses of fluid, and a dispenser head that is movable relative to said reservoir so as to dispense the fluid, in particular via a pump, a metering valve, or a piston that slides in said reservoir. When the user wishes to use the device, the user inserts the dispenser head into the nostril and actuates the device so as to dispense a dose of fluid, generally in the form of spray.

A drawback with prior-art devices relates to the effectiveness of the dose that is dispensed in the nostril, in particular when the purpose of the dispensed fluid is to act on the brain. Specifically, only a tiny portion of the dose generally reaches the target zone for this type of treatment, namely the olfactory zone including the ethmoid sinuses, in particular because of the orientation of the administering device in the nostril, which varies from one patient to another. Unfortunately, it appears that this orientation determines whether targeting of the target zone is successful, in particular for a compact spray that is used to obtain the maximum deposition in the target zone.

Document WO 2018/109409 describes a nasal dispenser assembly that associates a fluid dispenser device with a positioner member for positioning with a plurality of facial bearing zones. That assembly is not very discreet, is relatively bulky, and includes two separate sub-assemblies.

Documents WO 2006/106208 and FR 2 798 068 describe other prior-art devices.

An object of the present invention is to provide a nasal dispenser assembly that does not have the above-mentioned drawbacks.

In particular, an object of the present invention is to provide a nasal dispenser device that makes it possible to control the orientation of the device in the nostril.

Another object of the present invention is to provide a nasal dispenser device that improves the percentage of active fluid that is deposited on the olfactory zone and/or on the ethmoid sinuses.

Another object of the present invention is to provide a nasal dispenser device that is simple and inexpensive to manufacture and to assemble.

The present thus provides a nasal fluid dispenser device comprising: a body comprising a hollow sleeve and a top surface; and an actuator member that is mounted to slide in said body so as to actuate the device; the device further comprising two nasal extensions, at least one of which forms a dispenser head provided with a respective dispenser orifice, said nasal extensions extending from said top surface, each dispenser head being connected, during actuation, to a respective reservoir that is arranged in said body, said two nasal extensions forming an angle lying in the range 30° to 60° relative to a longitudinal central axis of said body.

Advantageously, said two nasal extensions are parallel.

Advantageously, said two nasal extensions form an angle lying in the range 40° to 50°, in particular about 45°.

Advantageously, said two nasal extensions have an insertion depth lying in the range 5 millimeters (mm) to 15 mm, in particular about 10 mm.

Advantageously, each reservoir contains a single dose of fluid for dispensing in a single actuation.

In a first advantageous variant, said fluid is a liquid, each reservoir comprising a hollow tube containing a piston, each dispenser head being connected to a respective needle, stationary in said body, for piercing said piston during actuation.

In a second advantageous variant, said fluid is a powder, each reservoir comprising a hollow tube including an air inlet at one end and a fluid outlet at the other end, said air inlet being provided with a fluid retainer member that is adapted to retain the powder in said reservoir before actuation and being connected to an air expeller that is formed by said actuator member, and said fluid outlet being closed before actuation by a closure element, such as a spherical ball, said air expeller being actuated manually by the user so as to create a flow of air that passes through said reservoir so as to deliver the powder contained therein towards the dispenser orifice(s).

Advantageously, the device includes a mechanical opening system that is secured to the air expeller and that is adapted to co-operate with said closure element so as to expel it mechanically from its closed position while the device is being actuated.

In a first advantageous embodiment, each of the two nasal extensions forms a dispenser head, each provided with a respective dispenser orifice.

Advantageously, the body contains two reservoirs, each of which is connected to a respective dispenser head during actuation, for dispensing fluid simultaneously into both nostrils.

In a second advantageous embodiment, one of the nasal extensions forms a dispenser head provided with a dispenser orifice for dispensing fluid into one of the two nostrils, and the other nasal extension forms a guide for co-operating with the other nostril.

Advantageously, a central portion of said top surface of said body, situated between said two nasal extensions, comes to bear against the septum of the nose during actuation.

Advantageously, said central portion has a profile that is slightly concave so as to adapt to the septum of the nose.

These and other characteristics and advantages appear more clearly from the following detailed description, given by way of non-limiting example, and with reference to the accompanying drawings, in which:

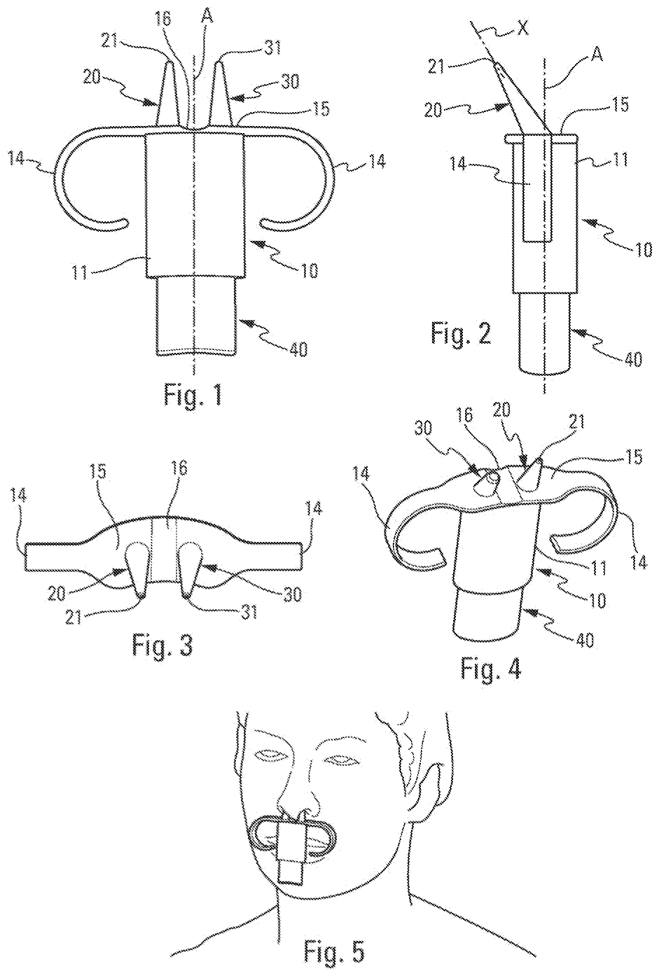

FIG. 1 is a diagrammatic front view in perspective of a nasal dispenser device in a first advantageous embodiment;

FIG. 2 is a diagrammatic side view in perspective of the FIG. 1 device;

FIG. 3 is a diagrammatic perspective view from above of the device in FIGS. 1 and 2;

FIG. 4 is a diagrammatic perspective view of a nasal dispenser device in a second advantageous embodiment;

FIG. 5 is a diagrammatic view showing a way of using the device in FIGS. 1 to 3;

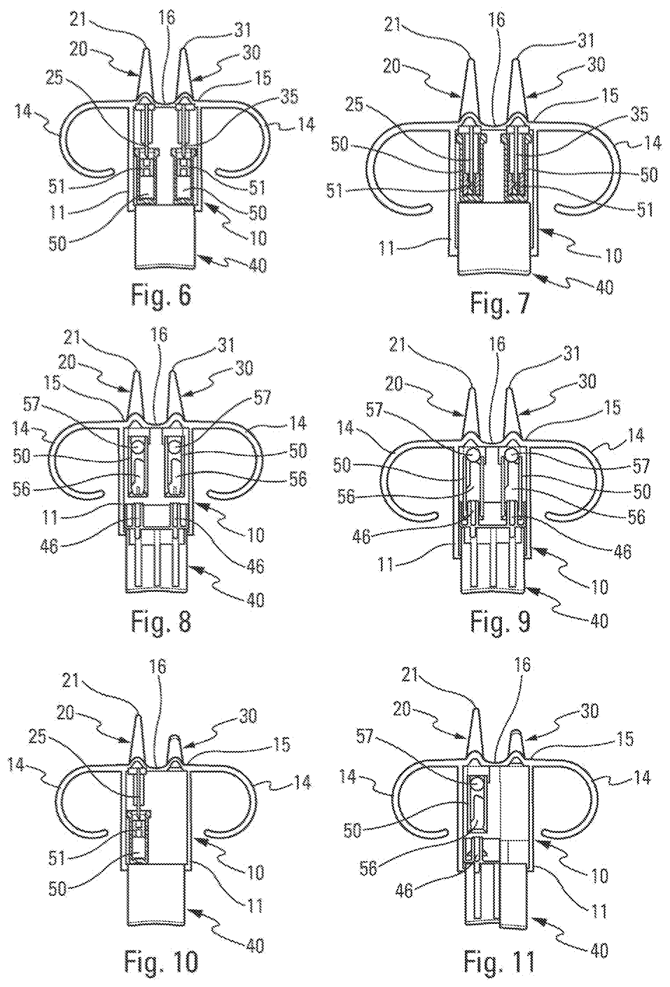

FIG. 6 is a diagrammatic section view of a nasal dispenser device in accordance with FIGS. 1 to 3, in a first advantageous variant embodiment, shown before use;

FIG. 7 is a view similar to the view in FIG. 6, shown after use;

FIG. 8 is a diagrammatic section view of a nasal dispenser device in accordance with FIGS. 1 to 3, in a second advantageous variant embodiment, shown before use;

FIG. 9 is a view similar to the view in FIG. 8, shown after use;

FIG. 10 is a diagrammatic section view of a nasal dispenser device in accordance with FIG. 4, in a first advantageous variant embodiment, shown before use; and

FIG. 11 is a diagrammatic section view of a nasal dispenser device in accordance with FIG. 4, in a second advantageous variant embodiment, shown before use.

The fluid dispenser device shown in the figures includes a body 10 on which there are secured two nasal extensions 20, 30, at least one of which is a nasal dispenser head. The nasal extensions 20, 30 extend from a top surface 15 of said body, and each is for penetrating into a respective nostril while the device is being used. Preferably, the axial extensions 20, 30 are relatively flexible so as to avoid any risk of injury while they are being inserted into the nostrils.

The central portion 16 of said top surface 15 of the body 10, which is situated between the two axial extensions 20, 30, comes to bear against the septum of the nose during actuation. Advantageously, the central portion 16 may have a profile that is slightly concave or curved so as to adapt to said septum, as can be seen in particular in FIG. 4. In a first advantageous embodiment, shown in

FIGS. 1 to 3 and 5 to 9, each of the two nasal extensions 20, 30 forms a nasal dispenser head, provided with a respectively dispenser orifice 21, 31. In this first embodiment, two variants of which are described in greater detail below, actuating the device causes fluid to be dispensed simultaneously into both nostrils.

In a second advantageous embodiment, shown in FIGS. 4, 10, and 11, one of the nasal extensions 20 forms a nasal dispenser head, provided with a dispenser orifice 21, and the other nasal extension 30 forms a guide. In this second embodiment, two variants of which are described in greater detail below, actuating the device causes fluid to be dispensed into a single nostril.

The two nasal extensions 20, 30 are sloping, i.e. the axis X along which the two axial extensions 20, 30 extend forms an insertion angle relative to the longitudinal axis A of the body 10, which longitudinal axis A is a vertical axis in the orientation in FIGS. 1, 2, and 6 to 11. Preferably, both nasal extensions 20, 30 are parallel.

The device of the invention makes it possible to orientate the dispenser head(s) in each nostril in a manner that is substantially predetermined, and thereby target the zones in the nostril(s) on which the fluid is to be dispensed.

Thus, the angle of insertion of each nasal extension 20, 30 in the respective nostril lies in the range 30° to 60°, advantageously in the range 40° to 50°, preferably about 45°.

In addition, the depth P of insertion of each dispenser head in the nostril is predetermined by the dimensions of the axial extensions 20, 30 relative to the top surface 15 of the body 10, and in particular relative to the central portion 16 that bears against the septum of the nose. The insertion depth advantageously lies in the range 5 mm to 15 mm, preferably about 10 mm.

The body 10 includes a hollow sleeve 11 that receives one or two reservoirs 50, as described below.

Advantageously, the body 10 also includes two external side loops 14 that are adapted to receive the user's fingers, in particular the index finger and the middle finger. In a variant, the user's fingers can press against the top surface 15, e.g. radially on either side of the two nasal extensions 20, 30.

An actuator member 40 is mounted to slide in the body 10 so as to actuate the device. Typically, the user's thumb is placed on the bottom of said actuator member 40, so as to perform actuation.

The device may be used to dispense a liquid as shown in FIGS. 6, 7, and 10, or a powder as shown in FIGS. 8, 9, and 11.

In a first advantageous embodiment, the device causes fluid to be dispensed simultaneously into both nostrils.

To do this, each of the two nasal extensions 20, 30 forms a nasal dispenser head, each being connected to a respective reservoir 50.

Preferably, each reservoir 50 contains a single dose of fluid for dispensing in a single actuation. The device is then known as a single-dose device, since all of the fluid is dispensed in a single actuation, even when respective doses are dispensed simultaneously into each nostril. Optionally, the present invention could be applied to multi-dose devices, e.g. two-dose devices.

FIGS. 6 and 7 show a first variant of the first embodiment in which the fluid is a liquid. In this variant, each reservoir 50 is fastened in the actuator member 40, and thus axially movable relative to the body 10 during actuation.

Each reservoir 50 is preferably in the form of a blind hollow tube that has a single axial opening that is closed by a piston 51 that forms a leaktight stopper before actuation. Each dispenser head is connected to a needle 25, 35 that is stationary relative to the body 10, and that, during actuation, pierces the respective piston 51 and enables the fluid contained in each reservoir 50 to be dispensed simultaneously through the dispenser orifices 21, 31 as each piston 51 slides in the respective reservoir 50.

FIGS. 8 and 9 show a second variant of the first embodiment in which the fluid is a powder. This second variant advantageously uses means similar to the means described in document WO 2015/001281.

In the second variant, each reservoir 50 is fastened in the body 10, and thus cannot move axially relative to the body 10 during actuation.

Each reservoir 50 is in the form of a hollow tube including an air inlet at one end and a fluid outlet at the other end. The air inlet of each reservoir is connected to an air expeller, and the fluid outlet of each reservoir is connected to a respective dispenser head. Before actuation, the fluid outlet is closed by a closure element 57, preferably a spherical ball, that is force fitted in said fluid outlet. The air inlet is provided with a fluid retainer member that is adapted to retain the powder in the reservoir before the device is actuated. The air expeller is formed by the actuator member 40 and is actuated manually by the user so as to create a flow of air that passes through each reservoir 50 so as to deliver the fluid contained therein towards the respective dispenser orifice 21, 31.

The device includes a mechanical opening system 46, 56 that is secured to the air expeller, i.e. it is actuated simultaneously with said air expeller being actuated, and that is adapted to co-operate with said closure element 57 so as to expel it mechanically from its closed position while the device is being actuated. The mechanical opening system may include a set of rods 46, 56, having a first rod portion 46 that is secured to the actuator member 40 forming the air expeller 20, and a second rod portion 56 that is arranged in the reservoir 50 and that is thrust by said first rod portion 46 when the device is actuated. At the end of their actuation stroke, the set of rods 46, 56 co-operates with the closure element 57 so as to expel it mechanically from its closed position.

The fluid retainer member may advantageously be made integrally with the second rod portion 56. Thus, the fluid retainer member can be made in leaktight and airtight manner before the device is actuated, the air pressure created by the air expeller penetrating into the reservoir 50 only when said retainer member is moved by being thrust by the first rod portion 46.

The air expeller, not shown in detail, comprises a piston, e.g. formed on the actuator member 40, sliding in an air chamber, e.g. formed in the sleeve 11 of the body 10, the piston being actuated manually by the user when the user moves the actuator member 40 relative to the body 10.

In a second advantageous embodiment, the device causes fluid to be dispensed into a single nostril.

To do this, only one of the nasal extensions 20, 30 forms a nasal dispenser head that is connected to a reservoir 50. The other nasal extension forms a guide that is for penetrating into the second nostril for the purpose of guiding and orientating the device. Preferably, the dispenser head and the guide are parallel, as can be seen in FIG. 4.

FIG. 10 shows a first variant of this second embodiment in which the fluid is a liquid. This variant is similar to the above-described variant in FIGS. 6 and 7.

FIG. 11 shows a second variant of this second embodiment in which the fluid is a powder. This variant is similar to the above-described variant in FIGS. 8 and 9.

The present invention thus provides a device that is simultaneously discreet, compact, easy to carry, intuitive to use, and with an orientation and an insertion depth that are appropriate and suited to the morphology of the user.

The present invention is particularly adapted for treatment of the central nervous system, such as treatment targeting neurodegenerative diseases of the Parkinson or Alzheimer type that require medication to be delivered via the nose-brain barrier. Reaching the target zone, in particular the ethmoid sinuses, via the nasal cavity is a simple and non-invasive means of administering a treatment to the brain. Other applications may be envisaged, such as the treatment of: autism; various forms of epilepsy; migraines; neurological diseases; attention deficit hyperactivity disorder (ADHD) or attention deficit disorder (ADD) without hyperactivity; dementia; multiple sclerosis; cerebrovascular diseases; brain tumors. The following molecules may be concerned: insulin; oxytocin; dopamine; levodopa; stem cell therapy (brain tumors); rasagiline mesylate; ropinirole; amantadine; sumatriptan; huperzine A; curcumin; geniposide; rivastigmine; rivastigmine tartrate; Doxepin; Agomelatine; fluoxetine hydrochloride; Tramadol hydrochloride; Nortriptyline hydrochloride; venlafaxine; paliperidone. For the treatment of Alzheimer: donepezil; galantamine; rivastigmine; memantine; and donepezil and memantine in combination.

The present invention is described above with reference to several advantageous embodiments and variants, but naturally any modification could be applied thereto by a person skilled in the art, without going beyond the ambit of the present invention, as defined by the accompanying claims.

1. A nasal fluid dispenser device comprising: a body (10) comprising a hollow sleeve (11) and a top surface (15); and an actuator member (40) that is mounted to slide in said body (10) so as to actuate the device; the device being characterized in that it further comprises two nasal extensions (20, 30), at least one of which forms a dispenser head provided with a respective dispenser orifice (21, 31), said nasal extensions extending from said top surface (15), each dispenser head being connected, during actuation, to a respective reservoir (50) that is arranged in said body (10), said two nasal extensions (20, 30) forming an angle lying in the range 30° to 60° relative to a longitudinal central axis (A) of said body (10).

2. A device according to claim 1, wherein said two nasal extensions (20, 30) are parallel.

3. A device according to claim 1, wherein said two nasal extensions (20, 30) form an angle lying in the range 40° to 50°, in particular about 45°.

4. A device according to claim 1, wherein said two nasal extensions (20, 30) have an insertion depth lying in the range 5 mm to 15 mm, in particular about 10 mm.

5. A device according to claim 1, wherein each reservoir (50) contains a single dose of fluid for dispensing in a single actuation.

6. A device according to claim 1, wherein said fluid is a liquid, each reservoir (50) comprising a hollow tube containing a piston (51), each dispenser head being connected to a respective needle (25, 35), stationary in said body (10), for piercing said piston (51) during actuation.

7. A device according to claim 1, wherein said fluid is a powder, each reservoir (50) comprising a hollow tube including an air inlet at one end and a fluid outlet at the other end, said air inlet being provided with a fluid retainer member that is adapted to retain the powder in said reservoir (50) before actuation and being connected to an air expeller that is formed by said actuator member (40), and said fluid outlet being closed before actuation by a closure element (57), such as a spherical ball, said air expeller being actuated manually by the user so as to create a flow of air that passes through said reservoir (50) so as to deliver the powder contained therein towards the dispenser orifice(s) (21, 31).

8. A device according to claim 7, including a mechanical opening system (46, 56) that is secured to the air expeller and that is adapted to co-operate with said closure element (57) so as to expel it mechanically from its closed position while the device is being actuated.

9. A device according to claim 1, wherein each of the two nasal extensions (20, 30) forms a dispenser head, each provided with a respective dispenser orifice (21, 31).

10. A device according to claim 9, wherein the body (10) contains two reservoirs (50), each of which is connected to a respective dispenser head during actuation, for dispensing fluid simultaneously into both nostrils.

11. A device according to claim 1, wherein one of the nasal extensions (20) forms a dispenser head provided with a dispenser orifice (21) for dispensing fluid into one of the two nostrils, and the other nasal extension (30) forms a guide for co-operating with the other nostril.

12. A device according to claim 1, wherein a central portion (16) of said top surface (15) of said body (10), situated between said two nasal extensions (20, 30), comes to bear against the septum of the nose during actuation.

13. A device according to claim 12, wherein said central portion (16) has a profile that is slightly concave so as to adapt to the septum of the nose.
